(19) United States
(12) Patent Application Publication
(10) Pub. No.: US 2004/0234580 A1
(43) Pub. Date: Nov. 25, 2004

(54) Animal feeds including actives and methods of using the same

(76) Inventors: Gordon R. Huber, Sabetha, KS (US); David R. Jones, Palm Beach, FL (US); Catherine M. Jones, Alpharetta, GA (US); John C. Kuenzi, Bern, KS (US); Kevin D. Kuenzi, Perry, KS (US); Francisco A. Cabrera, Overland Park, KS (US)

Correspondence Address: Hovey Williams, Suite 400, 2405 Grand Blvd, Kansas City, MO, 64108, US

(21) Appl. No.: 10/467,292

(22) PCT Filed: Oct. 7, 2002
(86) PCT No.: PCT/US02/32125

Related U.S. Application Data
(63) Continuation-in-part of application No. 09/971,521, filed on Oct. 5, 2001, now Pat. No. 6,716,448.
(63) Continuation-in-part of application No. 09/971,518, filed on Oct. 5, 2001, now abandoned.
(63) Continuation-in-part of application No. 09/971,519, filed on Oct. 5, 2001.
(63) Continuation-in-part of application No. 09/971,520, filed on Oct. 5, 2001.

(51) Int. Cl. A61K 31/7048; A23K 1/165

(52) U.S. Cl. 424/442; 514/28

(57) ABSTRACT

Improved daily ration mammal feeds are provided which include minor amounts of active such as heartworm preventative drugs, in order to ensure that a mammal consuming the feed receives quantities of active sufficient to establish and maintain substantially constant concentrations of the active in the pet's bloodstream. The feeds may be produced by methods including extrusion with addition of minor quantities of active therein so as to uniformly distribute the active throughout the product.

### BACKGROUND OF THE INVENTION

1. Field of the Invention

The present invention is broadly concerned with improved daily ration feed products for animals including minor amounts of an active or drug such as a pharmaceutical drug. More particularly, the invention is concerned with such feed products, and methods of preparing and using the products, wherein the feeds contain a sufficient quantity of an active or drug such as a heartworm preventative drug so that when the animals consume the feeds, therapeutically effective amounts of the active or drug are established and maintained in the bloodstreams of the animals. In this way, conventional dosing regimes are eliminated, and the animals receive proper quantities of the active as a part of their normal daily diets.

2. Description of the Prior Art

In recent years there has been a significant increase in animal research directed to determining proper nutritional standards and also effective drug treatments for animals. This is true not only in connection with domestic household pets such as dogs, cats, birds, and exotics, but also in regard to economically significant animals such as farm animals (e.g., horses, sheep and cattle) and zoo animals of all types.

Drug or active treatment of animals generally requires that these agents be administered from time to time by oral administration or injection, so that therapeutic amounts of the actives or drugs can be maintained in the bloodstreams of the animals either continuously or at least during a prescribed treatment period. Periodic dosing presents a number of difficulties. For example, the animal's caretaker may simply forget to administer a given drug or active at the required time. This can have the effect of disrupting a treatment protocol and even causing harm to the animal. For instance, dogs are conventionally treated with heartworm preventative drugs such as ivermectin on a monthly basis. If the dog's owner forgets to timely administer the drug, the dog is susceptible to heartworm infection. Another problem associated with periodic dosing of animals stems from the fact that the animals may be very reluctant to cooperate, especially if the drug or active is to be orally administered. Any cat owner can testify to the difficulty of persuading a domestic cat to consume a drug product.

A large number of actives can be used in the context of the invention, so long as the actives can withstand feed processing conditions and retain their potency. Among suitable actives are antibiotics, steroids, anti-inflammatory agents, endoectacides (e.g., dewormers such as heartworm-preventative drugs) and ectoparasiticides (e.g., drugs effective against fleas and ticks).

Heartworm infection is an endemic condition with certain animals, and especially household pets such as cats and dogs. A number of actives or drugs have been developed for the treatment of heartworm infection, such as the avermectins, which are a class of macrocyclic lactones. Drugs of this class include ivermectin, selamectin, moxidectin, milbemycin oxine and eprinomectin.

Ivermectin is a known oral and injectable medication used as a wormer, heartworm preventative and to kill certain mites (mange). Ivermectin is a mixture of (10E,14E,16E,22Z)-(1R,4S,5′S, 6S,6′R,8R, 12S,13S,20R,21R,24S)-6′-[(S)-sec-butyl]-21,24-dihydroxy-5′,11,13,22-tetramethyl-2-oxo-(3,7,19-trioxatetracyclo[15.6.1.1^{4,8}0^{20,24}]pentacosa-10,14,16,22-tetraene)-6-spiro-2′-(perhydropyran)-12-yl 2,6-dideoxy-4-O-(2,6-dideoxy-3-O-methyl-αa-L-arabino-hexopyranosyl)-3-O-methyl-αa-L-arabino-hexopyranoside and (10E, 14E, 16E,22Z)-(1R,4S,5′S,6S,6′R,8R,12S,13S,20R,21R,24S)-21,24-dihydroxy-6′-isopropyl-5′,11,13,22-tetramethyl-2-oxo-(3,7,19-trioxatetracyclo[15.6.1.1^{4,8}0^{20,24}]pentacosa-10,14,16,22-tetraene)-6-spiro-2′-(perhydropyran)-12-yl 2,6-dideoxy-4-O-(2,6-dideoxy-3-O-methyl-αa-L-arabino-hexopyranosyl)-3-O-methyl-αa-L-arabino-hexopyranoside CAS: #70288-86-7.

Selamectin is identified as (5Z,25S)-25-cyclohexyl-4′-O-de(2,6-dideoxy-3-O-methyl-αa-L-arabino-hexopyranosyl)-5-demethoxy-25-de(1-methylpropyl)-22, 23-dihydro-5-(hydroxyimino)avermectin A₁ₐ.

Moxidectin is SPIRO[11,15-METHANO-2H, 13H, 17H-FURO[4,3,2-PQ] [2,6]-B ENZODIOXACYCLO-OCTADECIN-13,2′[2H]PYRAN-17-ONE]-6′-[1,3-DIMETHYL-1-BUTENYL]-3′,4′,5′,6,6′,7,10,11,14,15,17a,20,20a,20b-DIHYDRO-4′-[METHOXYIMINO]-5′,6,6,19-TETRAMETHYL-[6R-2aE,4E,4E,5′S*,6R*,6′S*(E),8E, 11R*, 13R*, 15S*, 17aR*, 20R*, 20aR*, 20bS*]].

Milbemycin Oxime consists of the oxime derivatives of 5-didehydromilbemycins in the ratio of approximately 80% A4 (C32H45N07, MW 555.71) and 20% A3 (C31H43N07).

Eprinomectin is 4″-epiacetylamino-4″-deoxyavermectin B₁.

These drugs are conventionally provided in tablet form or, for larger animals, as pastes and injectable liquids. Generally, animals are treated with relatively large doses of these drugs on a periodic basis. In the case of dogs and cats, tablets/chewables are given once a month by mouth year round for heartworm prevention. Higher doses are used to eliminate other parasites.

Ivermectin is the most commonly used heartworm preventative drug in domestic pets, and is generally considered safe at recommended dosage levels. If these are exceeded, side effects such as tremors, staggering, dilated pupils, loss of body weight or death may occur. As a consequence of normal dosing regimes for ivermectin, the treated animals necessarily receive a relatively large quantity of the drug which is to remain effective for an extended period. This in turn means that shortly after treatment the animal has a very high concentration of ivermectin in its bloodstream, with this concentration tailing off during the remainder of the period. This is to be contrasted with a more preferable treatment protocol wherein a substantially constant level of ivermectin is maintained on a continuing basis.

By the same token, the other established heartworm preventative drugs are generally administered in the same fashion as ivermectin, i.e., a relatively large quantity of the drugs are given at intervals, rather than daily administration of the drug to achieve a maintenance level in the animal's bloodstream.

Attempts have been made in the past to provide daily ration products which include therapeutic drugs. For example, Hills Pet Food Products made and sold a Science Diet product referred to as “Maximum Stress Diet” which included small amounts of styrylpyridinium chloride and diethylcarbamazine in a canned dog food containing substantial quantities of animal fat which required refrigeration. However, the Maximum Stress Diet is no longer available, and was not optimum in that it required refrigeration and special handling. This is to be contrasted with conventional extruded feed products designed to be stored over extended periods at ambient temperature without significant loss of nutrients.

U.S. Pat. No. 6,190,591 describes a single-extruder process for the production of controlled release particles which maybe tableted. Various encapsulants including pharmaceuticals, nutraceuticals, nutritional compounds, biologically active components, flavorants, fragrances, detergents and surface-active compositions are described, at relatively large quantities in the particles of at least 1% and preferably from about 3-50%. Hence, the '591 patent is not concerned with complete feeds, but rather encapsulant particles. The process described in this patent makes use of an elongated extruder where water and lipid are successively injected into the barrel, followed by water evaporation from the barrel and final addition of encapsulants. Such equipment is generally not suited to the production of a daily ration feed or similar product, given the need to uniformly distribute an active in the latter type of product.

U.S. Pat. No. 5,550,153 describes methods for killing adult heartworms in dogs by the administration of ivermectin or similar drugs. The '153 patent teaches that such drugs may be incorporated into a canine feed ration. Nevertheless, the contemplated treatment regime is on a monthly basis, i.e., one dosage of ivermectin is given to the animal each month. Accordingly, this patent does not address the provision of a daily ration feed and the amount of ivermectin present in the once per month feeds is very high. Hence, the treatment regime according to this patent still suffers from the problem of delivering a very high concentration of drug immediately upon dosing, with a continual falloff of drug in the animal's bloodstream thereafter.

There is accordingly a need in the art for improved feeds including daily ration extrusion-processed feeds and methods of providing actives to animals in a manner which will avoid problems inherent in periodic dosing, while maintaining substantially constant therapeutic levels of actives such as ivermectin in the bloodstreams of the animals consuming the feeds on a daily basis.

### SUMMARY OF THE INVENTION

The present invention overcomes the problems outlined above and provides improved active containing daily ration feed products for animals such as cats, dogs, birds, exotics, horses, sheep, cattle, reptiles, other companion animals, and zoo animals and methods of preparing and using such feeds. Generally speaking, a wide variety of feed types can be improved in accordance with the invention, e.g., extrusion-processed feeds of either dry or semi-moist kind, canned/retorted feeds or fresh refrigerated feeds. When the feeds are produced by extrusion they contain respective quantities of protein, fat and starch, together with a relatively minor amount of a desired active or drug. Similarly, with canned or similar products a desired active is mixed with the solid and/or liquid fractions thereof to the desired therapeutic level. In all cases, however, it is preferred that the potency of the active content of the feeds be maintained for at least six months at ambient temperature storage, more preferably nine months, and most preferably from about nine to twenty-four months at ambient temperature storage.

As noted previously, a large number of actives can be used in the context of the invention, so long as the actives can withstand feed processing conditions and retain their potency. Among suitable actives are antibiotics, steroids, anti-inflammatory agents, endectocides (e.g., dewormers such as heartworm-preventative drugs) and ectoparasiticides (e.g., drugs effective against fleas and ticks).

Through use of the feed products of the invention, an animal consuming the feeds on a daily basis receives a maintenance quantity of the active, so that the therapeutic effects thereof are realized. Normally, the active should be present in the extruded feeds at a level of at least about 0.1 μg/kg of feed product more preferably from about 2-1500 μg/kg of feed product, and most preferably from about 4-1000 μg/kg of feed product, although specific active amounts may vary depending upon the particular active chosen. For example, ivermectin may be present at a level up to about 1000 μg/kg of feed product on a dry basis (db), more preferably from about 0.1-450 μg/kg offeed product (db), still more preferably from about 4-250 μg/kg of feed product (db), and most preferably from about 5-175 μg/kg of feed product (db). In other types of products within the ambit of the invention, the active may be present at a level of up to about 0.75% by weight, more preferably up to about 0.5% by weight, and still more preferably up to about 0.1% by weight. Preferably, the active content of the feeds should be maintained for a period of at least about six months, more preferably at least about nine months, and most preferably from about nine to twenty-four months, at ambient temperature storage conditions. Alternately, the amount of active present in the feeds should be sufficient to administer from about 0.025 to 5.35 μg active per kg of animal body weight per day, more preferably from about 0.050 to 3.0 μg active per kg of animal body weight per day, and most preferably from about 0.06 to 2.1 μg active per kg of animal body weight per day (assuming that each animal consumes 0.16 kg of the active-supplemented feed per kg of animal body weight per day).

As noted, a wide variety of extruded feeds can be used in the context of the invention. For example, typical dry extruded product having a moisture content of less than about 10% by weight can be produced with added active. Similarly, semi-moist feeds having a moisture content on the order of 15-30% by weight are also suitable. In extruded feeds of these types, it is preferred that the active content be substantially uniformly dispersed throughout the feed. Alternately, pillow-type feeds can be produced having a soft, flowable matrix center surrounded by a shell of self-sustaining feed material; in such a case, the active content may be present only in the soft center matrix. In most cases, the extruded feed products of the invention should contain from about 5-15% by weight moisture (wet basis), 15-30% by weight protein, more preferably from about 18-25% by weight protein; from about 3-24% by weight fat, more preferably from about 5-20% by weight fat; and from about 5-80% by weight starch, more preferably from about 20-50% by weight starch. Generally, the extruded feeds should have a bulk density of from about 30-700 g/l, more preferably from about 140-400 g/l, and a water activity of from about 0.1-0.99, more preferably from about 0.6-0.75.

An important goal of the invention is to provide active-containing daily ration feeds which when consumed on a daily basis by animals will establish and maintain a therapeutic amount of active in the bloodstreams of the animals. In this way, the need for periodic dosing with relatively large amounts of active(s) is completely avoided, yet the beneficial effects of the active remain. To this end, the feeds should have sufficient active therein so that, when an animal consumes the feed at a rate of from about 10-40 g of the feed per kg of the consuming animal's weight, the desired therapeutic amount of active is achieved.

During extrusion processing in accordance with the invention, starting farinaceous feed ingredients are fed into the elongated barrel of an extruder including at least one elongated, axially rotatable, helically flighted screw with an endmost extrusion die. During passage through the extruder barrel, the ingredients are subjected to elevated temperature, agitation and shear in order to cook the product. In preferred forms of the invention, the starting ingredients are first preconditioned prior to passage into the extruder barrel. Generally, during preconditioning the starting mixture is subjected to a temperature of from about 20-98° C. (more preferably from about 90-97° C.) for a period of from about 15-600 seconds (more preferably from about 170-190 seconds). The purpose of preconditioning is to initially moisturize and partially cook the starting material prior to entrance thereof into the extruder barrel. Advantageously, the material leaving the preconditioner has a moisture content of from about 10-60% by weight, and more preferably from about 21-23% by weight.

In the extruder, the preconditioned starting material is subjected to conditions of elevated heat, pressure and shear. Normally, the temperature conditions in the barrel are such as to achieve a maximum temperature of from about 20′-175° C., and more preferably from about 65-120° F. Normal maximum pressure conditions are from about 100-3000 psi, and more preferably from about 150-500 psi. Residence times in the extruder barrel usually range from about 3-180 seconds, and more preferably from about 20-40 seconds.

The active content of the extruded feeds can be added at a variety of locations during the process. One preferred technique is to prepare a dilute active solution which can be pumped at a known rate into the farinaceous ingredients during processing. For example, the active liquid may be added at the preconditioner, preferably adjacent the outlet thereof. Alternately, the active may be injected directly into the extruder barrel during processing. Given the relatively small quantities of active employed in the feeds, it is generally important that there be sufficient time in the process to adequately mix in the active substantially uniformly throughout the other ingredients.

It will be appreciated that the invention is not limited to extruded feed products, and that the principles of the invention can be applied with canned/retorted or fresh refrigerated animal foods. In addition, actives can be added to high moisture products (having a moisture content of from about 30-85% by weight). The types and contents of actives described above in connection with extruded feeds are equally applicable to such canned and fresh refrigerated feeds.

### DETAILED DESCRIPTION OF THE PREFERRED EMBODIMENTS

The following examples set forth presently preferred methods for the production of active-containing animal foods and related information. It is to be understood, however, that these examples are provided by way of illustration and nothing therein should be taken as a limitation upon the overall scope of the invention.

### EXAMPLE 1

In this example, an ivermectin-containing dog food product was produced using a co-extrusion process. The dry farinaceous ingredients used in this example were (all percentages on a weight basis): wheat flour-14%; rice flour-15%; corn flour-32%; corn gluten meal-12%; poultry meal-8%; brewer's yeast-2%; sodium bicarbonate-0.6%; Thoxyquin-0.1%; potassium sorbate-0.3%; and sugar-5%. The liquid co-extruded mixture contained (all percentages on a weightbasis: poultry fat-81.13%; GP (Glutamine Peptide)-1.32%; cheese powder-3.77%; and poultry meal-3.77%.

The extrusion equipment included a Wenger X-85 single screw extruder with a Wenger Model 7 DDC preconditioner. The extruder barrel was made up of a series of interconnected heads. The screw configuration, dies, adaptor parts, preconditioner shafts and beater elements were all Wenger equipment.

In order to effect the desired co-extrusion, a delivery pipe having approximately a ⅜″ delivery nipple was inserted into the center of the die so that the liquid portion was directed through the die with a surrounding annulus of the extruded farinaceous mixture. The liquid portion was pumped through the delivery pipe at a rate which was approximately 30% of the extrusion rate of the farinaceous mixture. At the outlet of the extruder die, the product was cut using an knife and respective samples of the cut product were manually crimped using a hand-crimping tool. In this fashion, “pillows” of the pet food were obtained, with an outer farinaceous ingredient shell and an inner flowable filling containing ivermectin.

Following this treatment, the product was dried to a moisture level of less than 10% by weight. Three samples from the dryer were subsequently frozen and another sample was placed in a plastic bag and stored at room temperature, for a period in excess of six months.

The following table sets forth the illustrative preconditioning and extrusion information.

TABLE 1
DRY RECIPE INFORMATION

| | | |
|---|---|---|
| Dry Recipe Rate | kg/hr | 93 |
| Feed Screw Seed | rpm | 11 |

PRECONDITIONING INFORMATION

| | | |
|---|---|---|
| Preconditioner Speed | rpm | 485 |
| Steam Flow to Preconditioner | kg/hr | 8 |
| Water Flow to Preconditioner | kg/hr | 21 |
| Preconditioner Discharge Temp. | ° C. | 66 |

EXTRUSION INFORMATION

| | | |
|---|---|---|
| Extruder Shaft Speed | rpm | 516 |
| Extruder Motor Load | % | 75 |
| Control/Temperature 2nd Head | ° C. | 40 |
| Control/Temperature 3rd Head | ° C. | 51 |
| Control/Temperature 4th Head | ° C. | 39 |
| Control/Temperature 5th Head | ° C. | 48 |
| Control/Temperature 7th Head | ° C. | 45 |

FINAL PRODUCT INFORMATION

| | | |
|---|---|---|
| Extruder Discharge Density | kg/m³ | 350 |

The products resulting from this test were analyzed to determine the content of ivermectin in the samples. In this analysis, each feed sample was ground in a Retsch mill at low speed using a 2 mm grating screen, so that the ground material would pass through a #10 mesh screen. A total of six samples, three frozen and three stored at room temperature, were processed. In each case, three 37.5 g of a sample was placed in a 250 ml bottle and 100 ml of methanol was added. The bottle was capped, the sample was sonicated for 20 minutes and shaken for 1 hour. 40 ml of the extract was added to a centrifuge tube and centrifuged for 5 minutes at 2000 rpm. 20 ml of the supernatant solution was then passed through a alumina column. The first five ml was rejected and the remainder of the liquid through the column was collected as a purified sample. 2 ml of the purified sample was mixed with a 5 ml mixture of acetonitrile:water (1:1), and a solid phase extraction (SPE) was performed in accordance with the procedure described in Doherty et al., Analytical Chemists International, 81:869(4) (1998). 2 ml of the working, 1% ivermectin sample standard was also run through the SPE procedure to determine if any loss of ivermectin was taking place.

All samples from the SPE treatment were evaporated under nitrogen using an analytical evaporator with a water bath temperature of 50° C. The dried samples were reconstituted in 2 ml of HPLC mobile phase for analysis. Two samples were also prepared using 2 ml of the working standard ivermectin solution (containing 0.42 μg/ml) and were run before and after the feed samples.

The HPLC setup consisted of the following:

Gilson 712 HPLC System Controller

Gilson 305 pump, 231 sample injector, 401 dilutor and 115 UV detector

Jones Chromatography column heater set at 30° C.

| | |
|---|---|
| HPLC Analytical column | Symmetry C₁₈, 5μ, 4.6 × 350 mm |
| Mobile Phase | Acetonitrile/methanol/water 53/35/7 |
| Flow rate | 1 mL/minute |
| UV Detection | 245 nm |

The results of the HPLC analyses (two injections of each feed sample and two injections of the working standard solution) confirmed that the pet food samples contained very close to the expected content (0.42 μg/kg) of ivermectin. In particular, the average ivermectin content of the three frozen and the ambient-stored samples was 0.43 μg/kg. This demonstrated that storage conditions (frozen versus ambient) had little effect upon ivermectin potency, and an excellent ivermectin stability.

### EXAMPLE 2

In this example, an ivermectin-containing dog food was prepared using a Wenger TX-85 twin screw extruder equipped with a Model 16 Wenger DDC preconditioner. The dry ingredients fed to the extruder included (all percentages by weight basis): wheat middlings-18%; meat and bone meal-18%; soybean meal-18%; and corn-46%. In this run, two liquid dispersions were used which contained (all percentages by weight basis): first mixture, propylene glycol-1 lbs and water-11 lbs; second mixture, propylene glycol-48.82%; water-48.82%; Red No. 40 dye-10.86%; and ivermectin solution-0.50%. The amount of ivermectin used was calculated to provide a dose of approximately 1121.1 μg of ivermectin per kg of the dog food on a dry basis.

The extruder barrel was made up of interconnected heads. The rotating elements within the barrel included extruder shafts and other elements. The extruder was equipped with dies and adaptors, inserts, and a cutting knife with knife blades was used. The foregoing components as well as the preconditioners shafts and beater elements were all Wenger equipment.

In the process, the dry ingredients were fed to the preconditioner where steam and water was added to moisturize and partially precook the mixture. This preconditioned material was then fed to the inlet of the extruder in the usual fashion. The first liquid mixture was added to the outlet end of the preconditioner for passage into the extruder barrel along with the preconditioned material, over a period of about 11 minutes. Thereafter, the colored, ivermectin-containing liquid mixture was added over a period of about 22 minutes. Finally, additional quantities of the first water/propylene glycol liquid mixture was again added, over about 11 minutes. After extrusion, the product was dried in a Wenger dryer operating at 115° C., with two drying passes of 7 and 8.9 minutes respectively, followed by a cooler pass with 4.5 minutes retention time. The dryer discharge moisture was 6.25%, wb.

Samples were collected of the colored ivermectin-containing dispersion, the raw material mixture, preconditioned material leaving the preconditioner and extruded samples.

The following table sets forth illustrative preconditioning and extrusion conditions.

TABLE 2
DRY RECIPE INFORMATION

| | | |
|---|---|---|
| Dry Recipe Moisture | % w b | 9.56 |
| Dry Recipe Density | kg/m³ | 570 |
| Dry Recipe Rate | kg/hr | 2618 |
| Feed Screw Speed | rpm | 205 |

PRECONDITIONING INFORMATION

| | | |
|---|---|---|
| Preconditioner Speed | rpm | 250 |
| Steam Flow to Preconditioner | kg/hr | 224 |
| Water Flow to Preconditioner | kg/hr | 362 |
| Preconditioner Additive 1 Rate | kg/hr | 57 |
| Preconditioner Discharge Temp. | ° C. | 90 |

EXTRUSION INFORMATION

| | | |
|---|---|---|
| Extruder Shaft Speed | rpm | 700 |
| Extruder Motor Load | % | 67 |
| Steam Flow to Extruder | kg/hr | 84 |
| Water Flow to Extruder | kg/hr | 112 |
| Control/Temperature 1st Head | ° C. | 50/57 |
| Control/Temperature 2nd Head | ° C. | 50/86 |
| Control/Temperature 3rd Head | ° C. | 40/52 |
| Control/Temperature 4th Head | ° C. | 40/75 |
| Head/Pressure | kPa | 900 |
| Knife Drive Speed | rpm | 905 |

FINAL PRODUCT INFORMATION

| | | |
|---|---|---|
| Extruder Discharge Density | kg/m³ | 368 |
| Extruder Performance | | Stable |

The dog food from this run was fed ad libitum to an intact female mixed breed dog weighing about 10 kg. On day 7, blood was drawn from the dog four hours after eating and stored in an anti-coagulant tube with calcium EDTA in a refrigerator. Seven days later, the same dog was again fed the ivermectin-containing feed ad libitum and blood was collected four hours post-feeding. This sample was also refrigerated in the same fashion as the first sample.

The blood samples were then analyzed to determine the content of ivermectin therein, using HPLC. The procedure used was described in Diclidnson, Journal of Chromatography, 58:250-257 (1990). In this procedure, 0.5 ml of each blood sample was purified using solid phase extraction (SPE) cartridges and dissolved in a small volume of mobile phase for injection onto the HPLC column. The method has a limit of detection of about 2 ng/ml and uses an internal standard. After preparation of the internal standard, a standard curve is constructed using ivermectin-spiked blood samples. A known 1% ivermectin sample was used as the primary standard.

The blood samples from the dog were then analyzed for ivermectin content with HPLC peak heights corrected using the internal standard. The HPLC setup consisted of the following:

Gilson 712 HPLC System Controller

Gilson 305 pump, 231 sample injector, 401 dilutor and 115 UV detector

Jones Chromatography column heater set at 56° C.

| | |
|---|---|
| HPLC Analytical column: | Coulter-Beckman |
| | UltraSphere XL C₁₈, 3μ, 4.6 × 70 mm |
| Mobile Phase: | Acetonitrile/methanol/water 49/33/18 |
| Flow Rate: | 1 mL/minute |
| UV Detection: | 245 nm |

The results of this study demonstrated that the dog blood samples contained ivermectin in the range of about 5-8 ng/ml.

### EXAMPLE 3

In this example a series of extrusion runs were performed to determine the consistency of metering of ivermectin into a dog food mixture during extrusion. In each case, the farinaceous mixture included the following ingredients (all percentages on a weight basis): corn-35.93%; poultry meal-28.94%; rice-22.95%; corn gluten meal-11.98%; vitamin premix-0.10%; and mineral premix-0.10%. Three ivermectin-containing liquids were prepared, containing: Recipe #1, propylene glycol-8.60 pounds; water-8.60 pounds; red #40 dye-160 grams; ivermectin solution-0.212 ml; Recipe #2, propylene glycol-8.60 pounds; water-8.60 pounds; red #40 dye-160 grams; ivermectin solution-0.433 ml; Recipe #3, propylene glycol-8.60 pounds; water-8.60 pounds; red #40 dye-160 grams; ivermectin solution-10.279 ml. In each run 8.0 kg of a respective ivermectin recipe was added to the farinaceous ingredients at the exit of the preconditioner, prior to entering the extruder barrel. The recipes were added at a rate equal to 2% of the farinaceous mixture rate. The target for the runs using Recipe #1 was 6 μg ivermectin/kg of feed; for runs using Recipe #2, 12 μg/kg; and for runs using Recipe #3, 36 μg/kg.

The extruder system employed was a Wenger model TX 57 twin screw extruder with a model 2 DDC preconditioner. The extruder barrel was equipped with an extrusion die, a knife assembly was used to cut extrudate.

The following table sets forth the preconditioning and extrusion information collected during this series of runs. In runs 101-103, Recipe #1 was used; in runs 104-106, Recipe #2 was used; and in runs 107-109, Recipe #3 was used. As the extrudates emerged from the die, they were cut using the knife assembly and dried in a Wenger multiple-pass drier. Samples were collected at 15 minutes, 30 minutes and 45 minutes from the preconditioner, extruder and drier.

TABLE 3

| | | | | | | | | |
|---|---|---|---|---|---|---|---|---|
| 101 | 102 | 103 | 104 | 105 | 106 | 107 | 108 | 109 |

DRY RECIPE INFORMATION:

| | | | | | | | | | | |
|---|---|---|---|---|---|---|---|---|---|---|
| Dry Recipe Density | kg/m³ | 494 | 494 | 494 | 494 | 494 | 494 | 494 | 494 | 494 |
| Dry Recipe Rate | kg/hr | 400 | 400 | 400 | 390 | 392 | 390 | 387 | 397 | 392 |
| Feed Screw Rate | rpm | 48 | 53 | 55 | 49 | 52 | 52 | 56 | 54 | 54 |

PRECONDITIONING INFORMATION:

| | | | | | | | | | | |
|---|---|---|---|---|---|---|---|---|---|---|
| Preconditioner | rpm | 350 | 350 | 350 | 350 | 350 | 350 | 350 | 350 | 350 |
| Speed | | | | | | | | | | |
| Steam Flow to | kg/hr | 36 | 35.8 | 35.9 | 36.1 | 35.9 | 35.8 | 36 | 36.1 | 35.9 |
| Preconditioner | | | | | | | | | | |
| Water Flow to Preconditioner | kg/hr | 48 | 48.1 | 48.3 | T47.7 | 47.9 | 48.1 | 48 | 48.2 | 48.1 |
| Preconditioner | kg/hr | 8 | 7.9 | 8.05 | 7.8 | 7.95 | 7.84 | 8.12 | 8.03 | 8.02 |
| Additive 1 Rate | | | | | | | | | | |
| Preconditioner Discharge Temp. | ° C. | 86 | 85 | 85 | 86 | 86 | 86 | 85 | 85 | 85 |
| Moisture Entering | % w b | 16.26 | 17.04 | 19.14 | 18.96 | 16.47 | 18.18 | 16.14 | 18.97 | 18.98 |
| Extruder | | | | | | | | | | |

EXTRUSION INFORMATION:

| | | | | | | | | | | |
|---|---|---|---|---|---|---|---|---|---|---|
| Extruder Shaft Speed | rpm | 426 | 427 | 425 | 427 | 426 | 426 | 426 | 426 | 425 |
| Extruder Motor Load | % | 53 | 45 | 61 | 54 | 52 | 67 | 49 | 51 | 52 |
| Steam Flow to | kg/hr | 12 | 13.1 | 709 | 8 | 7.9 | 8 | 8.1 | 8 | 8 |
| Extruder | | | | | | | | | | |
| Water Flow to | kg/hr | 24 | 24 | 24.1 | 24 | 24 | 23.8 | 24 | 24 | 23.9 |
| Extruder | | | | | | | | | | |
| Control/Temp. 1st Head | ° C. | 40/52 | 40/52 | 40/52 | 40/53 | 40/55 | 40/52 | 40/53 | 40/55 | 40/54 |
| Control/Temp. 2nd Head | ° C. | 60/60 | 60/60 | 60/59 | 60/60 | 60/60 | 60/59 | 60/59 | 60/59 | 60/60 |
| Control/Temp. 3rd Head | ° C. | 80/79 | 80/80 | 80/81 | 80/80 | 80/80 | 80/81 | 80/80 | 80/80 | 80/79 |
| Control/Temp. 4th Head | ° C. | 60/67 | 60/67 | 60/67 | 60/65 | 60/65 | 60/66 | 60/65 | 60/65 | 60/64 |
| Head/Pressure | kPa | 1710 | 1600 | 1980 | 1660 | 1770 | 1910 | 1960 | 1980 | 1830 |
| Knife Drive Speed | rpm | 1324 | 1324 | 1325 | 1492 | 1443 | 1493 | 1493 | 1492 | 1491 |

FINAL PRODUCT INFORMATION:

| | | | | | | | | | | |
|---|---|---|---|---|---|---|---|---|---|---|
| Extruder Discharge Moisture | % w b | 20.43 | 19.79 | 20.4 | 21.32 | 21.46 | 21.97 | 22.12 | 22.83 | 22.71 |
| Extruder Discharge Density | kg/m³ | 312 | 374 | 338 | 400 | 349 | 352 | 336 | 336 | 400 |
| Extruder Performance | | Stable | Stable | Stable | Stable | Stable | Stable | Stable | Stable | Stable |
| Dried Product Moisture | % w b | 2.75 | 2.12 | 4.67 | 9.38 | 9.74 | 10.18 | 7.45 | 9.4 | 8.0 |

The dried samples were analyzed to determine ivermectin content, using the technique described in Example 1. The results from the Recipe # 1, #2 and #3 runs were averaged, with the following results. For the Recipe # 1 runs (101-103), the ivermectin content was 6.02 μg/kg (db); for the Recipe #2 runs (104-106), the ivermectin content was 11.99 μg/kg (db); and for the Recipe #3 runs (107-109), the ivermectin content was 35.98 μg/kg (db). This confirms that the processing technique of this Example gives extremely close ivermectin contents, as compared with the pre-extrusion goals.

### EXAMPLE 4

In another series of tests, the methods described in Example 3 were followed to produce feed products containing 0.1, 0.2, 2, and 20 μg ivermectin per kg of feed product (db). The dried samples were analyzed to determine ivermectin content, using the technique described in Example 1. The final products contained 0.1, 0.2, 2, and 20 μg ivermectin per kg of feed product. This confirms that the processing technique of this Example gives extremely close ivermectin contents, as compared with the pre-extrusion goals

### EXAMPLE 5

In this example, a series of extrusion runs were carried out with dog food products containing different active ingredients. The equipment employed was a Wenger laboratory-scale X-5 extruder. The actives used in the respective runs were: Methoprene (insect growth regulator, Run 002); Lufenuron (insect growth regulator, chemically dissimilar to Methoprene, Run 003); Praziquantel (tapeworm treatment, Run 004); Enrofloxacin (potent broad spectrum antibiotic, Run 005); Dexamethasone (steroid of the cortisone type, Run 006); Ibuprofen (non-steroidal anti-inflammatory drug, Run 007); Fenbendazole (mammal dewormer, Run 008); Oxytetracycline (widely used antibiotic, Run 009); Ivermectin, Methoprene, Praziquantal cocktail (antiparasitical combination, Run 010); Imidaccopria (imidacloprid, Run 011); Amoxicillin (broad spectrum antibiotic, Run 012); Tribrissen (antibiotic, Run 013); Doramectin (broad spectrum dewormer and anthelmintic, Run 014).

In particular, the recipes for each run are set forth in the following table:

TABLE 4

| Recipe-Run 001 | By Weight |
|---|---|
| Corn | 35.9281% |
| Poultry Meal | 28.9421% |
| Rice | 22.9541% |
| Corn Gluten Meal | 11.9760% |
| Lasi Pet Premix | 0.0998% |
| Trace Mineral #95 | 0.0998% |
| Total | 100.0000% |

*0.980 kg of water was added to the above mixture

| Recipe-Run 002 | By Weight |
|---|---|
| Corn | 35.7982% |
| Poultry Meal | 28.8374% |
| Rice | 22.8711% |
| Corn Gluten Meal | 11.9327% |
| Lasi Pet Premix | 0.0994% |
| Trace Mineral #95 | 0.0994% |
| Hartz Methoprene Capsule Content | 0.3618% |
| Total | 100.0000% |

*0.980 kg of water was added to the above mixture
Calculated Active content in batch = 0.0051 kg

| Recipe-Run 003 | By Weight |
|---|---|
| Corn | 35.7982% |
| Poultry Meal | 28.8374% |
| Rice | 22.8711% |
| Corn Gluten Meal | 11.9327% |
| Lasi Pet Premix | 0.0994% |
| Trace Mineral #95 | 0.0994% |
| Lufenuron-Novartis | 0.01571% |
| Total | 99.7954% |

*0.980 kg of water was added to the above mixture
Calculated Active content in batch = 0.0053 kg

| Recipe-Run 004 | By Weight |
|---|---|
| Corn | 35.2250% |
| Poultry Meal | 28.3757% |
| Rice | 22.5049% |
| Corn Gluten Meal | 11.7417% |
| Lasi Pet Premix | 0.0978% |
| Trace Mineral #95 | 0.0978% |
| Bayer Droncit (Praziquantel) | 1.0176% |
| Propylene Glycol | 0.1571% |
| Total | 100.0000% |

*0.880 kg of water was added to the above mixture
Calculated Active content in batch = 0.0028 kg

| Recipe-Run 005 | By Weight |
|---|---|
| Corn | 35.5731% |
| Poultry Meal | 28.6561% |
| Rice | 22.7273% |
| Corn Gluten Meal | 11.8577% |
| Lasi Pet Premix | 0.0988% |
| Trace Mineral #95 | 0.0988% |
| Bayer Baytril Injectable (Enfloxacin) | 0.9881% |
| Total | 100.0000% |

*0.930 kg of water was added to the above mixture
Calculated Active content in batch = 0.0050 kg

| Recipe-Run 006 | By Weight |
|---|---|
| Corn | 35.6679% |
| Poultry Meal | 26.3158% |
| Rice | 20.8711% |
| Corn Gluten Meal | 10.8893% |
| Lasi Pet Premix | 0.0907% |
| Trace Mineral #95 | 0.0907% |
| Dexamethasone Solution | 9.0744% |
| Total | 100.0000% |

*0.480 kg of water was added to the above mixture
Calculated Active content in batch = 0.0010 kg

| Recipe-Run 007 | By Weight |
|---|---|
| Corn | 35.8728% |
| Poultry Meal | 28.8975% |
| Rice | 22.9187% |
| Corn Gluten Meal | 11.9576% |
| Lasi Pet Premix | 0.0996% |
| Trace Mineral #95 | 0.0996% |
| Ibuprofen | 0.1541% |
| Total | 100.0000% |

*0.980 kg of water was added to the above mixture
Calculated Active content in batch = 0.0050 kg

| Recipe-Run 008 | By Weight |
|---|---|
| Corn | 35.5554% |
| Poultry Meal | 28.6419% |
| Rice | 22.7160% |
| Corn Gluten Meal | 11.8518% |
| Lasi Pet Premix | 0.0988% |
| Trace Mineral #95 | 0.0988% |
| Pavacur-Febendzole Paste | 01.0374% |
| Total | 100.0000% |

*0.980 kg of water was added to the above mixture
Calculated Active content in batch = 0.005052 kg

| Recipe-Run 009 | By Weight |
|---|---|
| Corn | 35.5731% |
| Poultry Meal | 28.6561% |
| Rice | 22.7273% |
| Corn Gluten Meal | 11.8577% |
| Lasi Pet Premix | 0.0988% |
| Trace Mineral #95 | 0.0988% |
| Maxim 200-Oxytetracycline Solution | 0.9881% |
| Total | 100.0000% |

*0.930 kg of water was added to the above mixture
Calculated Active content in batch = 0.0050 kg

| Recipe-Run 010 | By Weight |
|---|---|
| Corn | 35.4801% |
| Poultry Meal | 28.5812% |
| Rice | 22.6678% |
| Corn Gluten Meal | 11.8267% |
| Lasi Pet Premix | 0.0986% |
| Trace Mineral #95 | 0.0986% |
| Equvalan Paste-Ivermectin | 0.1344% |
| Hartz Methoprene Capsule Content | 0.1271% |
| Bayer Droncit (Praziquantel) | 0.9856% |
| Total | 100.0000% |

*0.980 kg of water was added to the above mixture
Calculated Ivermectin Active content in batch = 0.000114 kg
Calculated Methoprene Active content in batch = 0.005082 kg
Calculated Praziquantel Active content in batch = 0.00284 kg

| Recipe-Run 011 | By Weight |
|---|---|
| Corn | 35.7001% |
| Poultry Meal | 28.7584% |
| Rice | 22.8084% |
| Corn Gluten Meal | 11.9000% |
| Lasi Pet Premix | 0.0992% |
| Trace Mineral #95 | 0.0992% |
| Bayer Advantage-Imidacloprid | 0.6347% |
| Total | 100.0000% |

*0.948 kg of water was added to the above mixture
Calculated Active content in batch = 0.002912 kg

| Recipe-Run 012 | By Weight |
|---|---|
| Corn | 35.8905% |
| Poultry Meal | 28.9118% |
| Rice | 22.9300% |
| Corn Gluten Meal | 11.9635% |
| Lasi Pet Premix | 0.0997% |
| Trace Mineral #95 | 0.0997% |
| Amoxicillin-Antibiotic | 0.1049% |
| Total | 100.0000% |

*0.980 kg of water was added to the above mixture
Calculated Active content in batch = 0.005 kg

| Recipe-Run 013 | By Weight |
|---|---|
| Corn | 35.8802% |
| Poultry Meal | 28.9035% |
| Rice | 22.9234% |
| Corn Gluten Meal | 11.9601% |
| Lasi Pet Premix | 0.0997% |
| Trace Mineral #95 | 0.0997% |
| Tribrissen-Antibiotic | 0.1336% |
| Total | 100.0000% |

*0.980 kg of water was added to the above mixture
Calculated Active content in batch = 0.00576 kg

| Recipe-Run 014 | By Weight |
|---|---|
| Corn | 35.8566% |
| Poultry Meal | 28.8845% |
| Rice | 22.9084% |
| Corn Gluten Meal | 11.9522% |
| Lasi Pet Premix | 0.0996% |
| Trace Mineral #95 | 0.0996% |
| Doramectin (Dectomax) | 0.1992 |
| Total | 100.0000% |

*0.970 kg of water was added to the above mixture
Calculated Active content in batch = 0.0001 kg

The X-5 extruder included seven interconnected heads with a single extruder shaft supporting rotating elements. The X-5 was also equipped with a Wenger die/adaptor. The extrudates were manually cut upon emerging from the die and were dried in a laboratory drier to a moisture content less than 10% by weight.

In each run the active ingredient(s) were diluted into a miscible liquid (water or propylene glycol) and combined with 0.5 kg of the recipe to make a premix. This premix was then loaded into a Hobart mixer along with the remaining contents of the batch (total of 5 kg in each case) and mixed to obtain the final recipe for extrusion. The individual batches were loaded into the feeding bin and the extrusion runs were started. Samples were taken at regular intervals of approximately 5 minutes after stable conditions were achieved. Some samples were taken “as is” from the extruder without drying and were frozen. Other dried samples were bagged and maintained at ambient temperature.

We claim:

1. A daily ration animal feed including therein a quantity of an active, said active being present in said feed at a level of at least about 0.1 μg drug/kg of feed.

2. The feed of claim 1, said active being selected from the group consisting of antibiotics, steroids, anti-inflammatory agents, endectocides, ectoparasiticides and mixtures thereof.

3. The feed of claim 2, said active being an endectocide selected from the group consisting of the avermectin class of drugs

4. The feed of claim 3, said active selected from the group consisting of ivermectin, selamectin, moxidectin, milbemycin oxine and eprinomectin, and mixtures thereof.

5. The feed of claim 1, said level being from about 2-1500 μg active/kg feed.

6. The feed of claim 1, said level being from about 2-450 μg active/kg feed.

7. The feed of claim 1, said active being substantially uniformly dispersed throughout the feed.

8. The feed of claim 1, said feed product containing respective quantities of protein, fat and starch and being selected from the group consisting of extruded feed products, canned feed products and fresh refrigerated feed products.

9. The feed of claim 8, said extruded feed product selected from the group consisting of dry and semi-moist extruded feed products.

10. The feed of claim 1, said active being present at a level so that, when an animal consumes the feed at a daily rate of from about 10-40 g feed per kg of the animal's weight, a therapeutically effective amount of the active is established and maintained in the animal's bloodstream.

11. A method of feeding an animal comprising the steps of feeding the animal on a daily basis an extrusion processed daily ration containing a quantity of an active, said active present in the ration so that a therapeutically effective amount of the active is established and maintained in the animal's bloodstream.

12. The method of claim 11, said active being selected from the group consisting of antibiotics, steroids, anti-inflammatory agents, endectocides, ectoparasiticides and mixtures thereof.

13. The method of claim 12, said active being an endectocide selected from the group consisting of the avermectin class of drugs.

14. The method of claim 11, said active being present in said feed at a level of at least about 0.1 μg active/kg of feed.

15. The method of claim 14, said level being from about 2-1500 μg active/kg feed.

16. The method of claim 11, said active being substantially uniformly dispersed throughout the feed.

17. The method of claim 11, said feed being an extruded feed product containing respective quantities of protein, fat and starch.

18. The method of claim 17, said extruded feed product selected from the group consisting of dry and semi-moist extruded feed products.

19. The method of claim 11, said active being present at a level so that, when the animal consumes the feed at a daily rate of from about 10-40 g feed per kg of the animal's weight, a therapeutically effective amount of the active is established and maintained in the animal's bloodstream.

20. The method of claim 11, said animal subject to heartworm infection and said active being selected from the avermectin class of drugs.

21. The method of claim 20, said active being selected from the group consisting of ivermectin, selamectin, moxidectin, milbemycin oxine and eprinomectin, and mixtures thereof.

22. The method of claim 21, said active being present in said feed at a level of at least about 0.1 μg drug/kg of feed.

23. The method of claim 22, said level being from about 0.1-1500 μg drug/kg feed.

24. In a method of producing an extruded daily ration feed product for animals including the steps of providing a mixture of ingredients including respective quantities of protein, fat and starch, passing said mixture into and through an elongated extruder barrel having an elongated, axially rotatable, helically flighted screw within the barrel and an endmost extrusion die in order to subject the mixture to elevated temperature, pressure and shear and thereby cook the mixture, the improvement which comprises the step of adding a quantity of an active to said mixture prior to passage thereof through said die.

25. The method of claim 24, including the step of initially passing said mixture through a preconditioner prior to passage thereof into and through said extruder, and, during said passage through the preconditioner, adding moisture to the mixture and elevating the temperature thereof to at least partially precook the mixture.

26. The method of claim 25, including the step of adding said active into said preconditioner.

27. The method of claim 26, including the step of providing said active in the form of a liquid, and injecting said liquid into said preconditioner adjacent the outlet thereof.

28. The method of claim 24, including the step of injecting said active into the barrel of said extruder during passage of the material into and through the barrel.

29. The method of claim 24, including the step of adding sufficient active to the mixture so that, when an animal consumes the feed on a daily basis, a therapeutically effective concentration of the active is established and maintained in the bloodstream of said animal.

30. The method of claim 24, including the step of adding sufficient active to the mixture so that the extruded feed has from about 0.1-1500 μg drug/kg of feed.

31. The method of claim 24, said active being selected from the group consisting of antibiotics, steroids, anti-inflammatory agents, endectocides, ectoparasiticides and mixtures thereof

32. The method of claim 31, said active being an endectocide selected from the group consisting of the avermectin class of drugs.

33. The method of claim 32, said active selected from the group consisting of ivermectin, selamectin, moxidectin, milbemycin oxine and eprinomectin, and mixtures thereof.

34. The method of claim 24, said quantity of active being up to about 0.75% by weight, based upon the total weight of the feed product taken as 100% by weight.

35. An animal feed including therein up to about 0.75% by weight of an active, based upon the total weight of the drug taken as 100%.

36. The feed of claim 35, said active selected from the group consisting of antibiotics, steroids, anti-inflammatory agents, endectocides, ectoparasiticides and mixtures thereof.

37. The feed of claim 36, said active being an endectocide selected from the group consisting of the avermectin class of drugs.

38. The feed of claim 37, said avermectin being selected from the group consisting of ivermectin, selamectin, moxidectin, milbemycin oxine and eprinomectin, and mixtures thereof.

39. A feed for animals comprising an extruded feed product containing respective quantities of protein, fat and starch, said extruded product feed having a quantity of ivermectin therein.

40. The feed of claim 39, said ivermectin quantity being effective for continuously maintaining in the bloodstream of an animal consuming the feed on a daily basis a therapeutic amount of ivermectin.

41. The feed of claim 39, said ivermectin being present in the extruded product feed at a level of at least about 0.1 μg ivermectin/kg of the extruded product feed.

42. The feed of claim 41, said level being from about 2-1500 μg/kg of extruded product feed.

43. The feed of claim 39, said ivermectin being substantially uniformly dispersed throughout the extruded product feed.

44. The feed of claim 39, said ivermectin being within a soft, flowable matrix, said matrix surrounded by a shell of self-sustaining edible feed material.

45. The feed of claim 39, said extruded product feed being selected from the group consisting of dry and semi-moist extruded product feeds.

46. The feed of claim 39, said extruded product feed having sufficient ivermectin therein so that, when an animal consumes the feed at a rate of from about 10-40 g of extruded feed product per kg of the animal's weight, said therapeutically effective amount is achieved.

47. The feed of claim 39, said extruded product feed being a dry food product and having a moisture content of less than about 10% by weight.

48. The feed of claim 39, said extruded product being a semi-moist food product and having a moisture content of from about 15-30% by weight.

49. The feed of claim 39, said animals selected from the group consisting of dogs and cats.

50. The feed of claim 39, said quantity of ivermectin being up to about 0.75% by weight ivermectin therein, based upon the total weight of the product taken as 100% by weight.

51. In a method of producing an extruded feed product for animals including the steps of providing a mixture of ingredients including respective quantities of protein, fat and starch, passing said mixture into and through an elongated extruder barrel having an elongated, axially rotatable, helically flighted screw within the barrel and an endmost extrusion die in order to subject the mixture to elevated temperature, pressure and shear and thereby cook the mixture, the improvement which comprises the step of adding a quantity of an active to said mixture prior to passage thereof through said die.

52. The method of claim 51, including the step of initially passing said mixture through a preconditioner prior to passage thereof into and through said extruder, and, during said passage through the preconditioner, adding moisture to the mixture and elevating the temperature thereof to at least partially precook the mixture.

53. The method of claim 52, including the step of adding said active into said preconditioner.

54. The method of claim 52, including the step of providing said active in the form of a liquid, and injecting said liquid into said preconditioner adjacent the outlet thereof.

55. The method of claim 51, including the step of injecting said active into the barrel of said extruder during passage of the material into and through the barrel.

56. The method of claim 51, including the step of adding sufficient active to the mixture so that, when an animal consumes the feed on a daily basis, a therapeutically effective concentration of active is established and maintained in the bloodstream of said animal.

57. The method of claim 51, including the step of adding sufficient active to the mixture so that the extruded feed has from about 0.1-1500 μg ivermectin/kg of feed.

58. The method of claim 51, including the step of adding sufficient active to the mixture so that the extruded feed has up to about 0.75% by weight active therein, based upon the total weight of the extruded feed taken as 100% by weight.

59. The method of claim 51, said active selected from the group consisting of antibiotics, steroids, anti-inflammatory agents, endectocides, ectoparasiticides and mixtures thereof.

60. The method of claim 59, said active being an endectocide selected from the group consisting of the avermectin class of drugs.

61. The method of claim 60, said avermectin being selected from the group consisting of ivermectin, selamectin, moxidectin, milbemycin oxine and eprinomectin, and mixtures thereof.

62. A daily ration animal feed comprising an extruded feed product containing respective quantities of protein, fat, and starch, said extruded feed product having a quantity of at least one active therein effective for continuously maintaining in the bloodstream of the animal consuming the feed on a daily basis a therapeutic amount of the active.

63. The feed of claim 62, said active selected from the group consisting of antibiotics, steroids, anti-inflammatory agents, endectocides, ectoparasiticides and mixtures thereof.

64. The feed of claim 62, said active being present in the extruded product feed at a level of at least about 0.1 μg active/kg of the extruded product feed.

65. The feed of claim 62, said level being from about 0.1-1500 μg active/kg feed.

66. The feed of claim 62, said drug being substantially uniformly dispersed throughout the feed.

67. The feed of claim 62, said extruded feed product selected from the group consisting of dry and semi-moist extruded feed products.

68. The feed of claim 62, said active being present at a level so that, when an animal consumes the feed at a daily rate of from about 10-40 g feed per kg of the animal's weight, a therapeutically effective amount of the active is established and maintained in the animal's bloodstream.

69. In a method of producing an extruded daily ration feed product for animals including the steps of providing a mixture of ingredients including respective quantities of protein, fat and starch, passing said mixture into and through an elongated extruder barrel having an elongated, axially rotatable, helically flighted screw within the barrel and an endmost extrusion die in order to subject the mixture to elevated temperature, pressure and shear and thereby cook the mixture, the improvement which comprises the step of adding a quantity of at least one active to said mixture prior to passage thereof through said die, said active present at a level effective for continuously maintaining in the bloodstream of the animal consuming the feed on a daily basis a therapeutic amount of the active.

70. The method of claim 69, including the step of initially passing said mixture through a preconditioner prior to passage thereof into and through said extruder, and, during said passage through the preconditioner, adding moisture to the mixture and elevating the temperature thereof to at least partially precook the mixture.

71. The method of claim 70, including the step of adding said active into said preconditioner.

72. The method of claim 71, including the step of providing said active in the form of a liquid, and injecting said liquid into said preconditioner adjacent the outlet thereof.

73. The method of claim 69, including the step of injecting said active into the barrel of said extruder during passage of the material into and through the barrel.

74. The method of claim 69, including the step of adding sufficient active to the mixture so that the extruded feed has from about 0.1-1500 μg active/kg of feed.

75. The method of claim 69, said active selected from the group consisting of antibiotics, steroids, anti-inflammatory agents, endectocides, ectoparasiticides and mixtures thereof.

76. An animal feed comprising an extruded feed product containing respective quantities of protein, fat, and starch, said extruded feed product having a quantity of at least one active therein present at a level of up to about 0.75% by weight, based upon the total weight of the feed taken as 100% by weight.

77. The feed of claim 76, said active quantity being effective for continuously maintaining in the bloodstream of the animal consuming the feed on a daily basis a therapeutic amount of the active.

78. A method of feeding an animal comprising the step of feeding an animal the feed of claim 76.
